(19) United States
(12) Patent Application Publication
(10) Pub. No.: US 2024/0247115 A1
(43) Pub. Date: Jul. 25, 2024

(54) MULTI-FUNCTIONAL CONDUCTING POLYMER COATING ON VASCULAR ACCESS DEVICES

(71) Applicant: Becton, Dickinson and Company, Franklin Lakes, NJ (US)

(72) Inventor: Leandro Forciniti, Wharton, NJ (US)

(73) Assignee: Becton, Dickinson and Company, Franklin Lakes, NJ (US)

(21) Appl. No.: 18/415,889

(22) Filed: Jan. 18, 2024

Related U.S. Application Data
(60) Provisional application No. 63/440,510, filed on Jan. 23, 2023.

(51) Int. Cl. C08J 5/18; A61L 29/08; A61L 29/16; A61L 33/00; C08F 299/00

(52) U.S. Cl. CPC C08J 5/18; A61L 29/085; A61L 29/16; A61L 33/0011; A61L 33/0094; C08F 299/00; A61L 2300/236; A61L 2300/42; C08J 2353/00

(57) ABSTRACT

Provided are conductive films including a heparin modified polymer. The heparin modified polymer comprises heparin covalently bound to a beta-substituted monomer having a conjugated backbone that is co-polymerized to its non-functionalized pair. The heparin modified polymer on a conductive film can be manufactured by co-polymerizing a beta-substituted monomer with a non-functionalized monomer to form a polymer on a conductive film. The polymer is reacted with N,N′-Dicyclohexylcarbodiimide (DCC) and acetic acid to form an anhydride of the polymer on the conductive film. The anhydride is then reacted with heparin to form the heparin modified polymer on the conductive film. Medical devices having a coating comprising the conductive film are also described.

### CROSS-REFERENCE TO RELATED APPLICATION

This application claims priority to U.S. Provisional Application No. 63/440,510, filed Jan. 23, 2023, the entire disclosure of which is hereby incorporated by reference herein.

### TECHNICAL FIELD

Embodiments of the disclosure generally relate to medical devices and methods of manufacture. More particularly, embodiments of the disclosure are directed to a heparin modified polymer, conductive films comprising the heparin modified polymer, medical devices having the conductive film coated thereon, and methods of manufacturing the heparin modified polymer.

### BACKGROUND

Infusion therapy medical devices, such as syringe cannulas and catheters used for sampling or medicament administration, typically have components that are in direct contact with infusion fluid and/or bodily fluid that can cause infection. For example, catheter-related bloodstream infections may be caused by colonization of microorganisms, which can occur in patients whose treatment includes intravascular catheters and I.V. access devices. These infections can lead to illness and excess medical costs. Impregnating and/or coating catheters and I.V. access devices with various antimicrobial agents (e.g., chlorhexidine, silver, or other antibiotics) is a common approach that has been implemented to prevent these infections.

Some blood contact devices have the potential to generate thrombus. When blood contacts a foreign material, a complex series of events occur. These involve protein deposition, cellular adhesion and aggregation, and activation of blood coagulation schemes. Thrombogenicity has conventionally been counteracted by the use of anticoagulants, such as heparin. Attachment of heparin to otherwise thrombogenic polymeric surfaces may be achieved with various surface coating techniques.

Electrically conducting polymers have been used for tissue engineering materials because they are capable of delivering electrical cues to target sites and can simultaneously provide physical support for cell growth. They have also been used for deep neural brain stimulation and biosensors. Heparin is known to act as an anti-coagulant by binding to the enzyme inhibitor antithrombin III (AT) causing a conformational change that results in its activation through an increase in the flexibility of its reactive site loop. The activated AT then inactivates thrombin, factor Xa, and other proteases. It has been shown that bound heparin on biomaterials surfaces are effective in inhibiting blood clots.

Previous technologies to integrate sensors into catheters have been focused on using fiber optics within the lumen of the catheter or physical pressure transducers and not printed electronics.

Thus, there is a need for medical devices and methods that can achieve antithrombogenic characteristics and eliminate the major difficulties of printed electronics on vascular access devices.

### SUMMARY

One or more embodiments are directed a conductive film comprising. The conductive film comprises a heparin modified polymer, the heparin modified polymer comprising heparin covalently bound to a beta-substituted monomer having a conjugated backbone that is co-polymerized to its non-functionalized pair.

Embodiments of the disclosure are directed to a medical device having a coating comprising the conductive film of one or more embodiments.

An additional embodiment is directed to method of manufacturing a heparin modified polymer on a conductive film. In one or more embodiments, the method comprises: co-polymerizing a beta-substituted monomer with a non-functionalized monomer to form a polymer on a conductive film; reacting the polymer with N,N′-Dicyclohexylcarbodiimide (DCC) and acetic acid to form an anhydride of the polymer on the conductive film; and reacting the anhydride with heparin to form the heparin modified polymer on the conductive film.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a plan view of an exemplary medical device; and

FIG. 2 is at FTIR spectra of conductive films according to the examples.

To facilitate understanding, identical reference numerals have been used, where possible, to designate identical elements that are common to the figures. The figures are not drawn to scale and may be simplified for clarity. Elements and features of one embodiment may be beneficially incorporated in other embodiments without further recitation.

### DETAILED DESCRIPTION

Before describing several exemplary embodiments of the invention, it is to be understood that the invention is not limited to the details of construction or process steps set forth in the following description. The invention is capable of other embodiments and of being practiced or being carried out in various ways.

The following terms shall have, for the purposes of this application, the respective meanings set forth below.

An antithrombogenic agent is a substance which prevents the formation of a blood clot. In one or more embodiments, an antithrombogenic agent can be coated on a device to prevent the formation of a blood clot.

Principles and embodiments of the present disclosure relate generally to medical devices having improved properties, and methods of preparing and using them. Provided are medical articles, for example, catheter tubing, which have antithrombogenic properties. conductive films comprising a heparin modified polymer that can be coated on a medical device, e.g., catheter, extensions, IV tubing, catheter adapter, Luer port, connector body, device housing, a component thereof, combinations thereof, and the like, could be patterned onto a soft polymer substrate or composite polymer substrate typically used for vascular access such as peripherally inserted central catheter (PICC), peripherally intravenous catheter (PIVC), central venous catheter (CVC), arterial line catheters, implanted ports or implanted in the vasculature such as heart valves or endovascular stents, and the like, in order to prevent blood stream infections and blood clots, such as deep vein thrombosis (DVT) and thrombosis-induced catheter occlusions. In one or more embodiments, either a pad containing this hybrid conducting substrate would exist at the distal end of the catheter or along the catheter length electrically addressed by either a conductive strip of conducting polymer or other electrically conductive metals printed or patterned lengthwise along the catheter.

Heparin, also known as unfractionated heparin (UFH), is a pharmaceutical and naturally occurring glycosaminoglycan with a negative ionic charge. As recognized by one of skill in the art, heparins depend on the activity of antithrombin, so they are considered anticoagulants. In its natural unfractionated state, heparin exists as a heterogeneous mixture of oligosaccharides composed of alternating chains of D-glucosamine and uronic acid. The predominant anticoagulant property of heparin is based on its interaction with antithrombin (AT) (formerly known as antithrombin III). This interaction is dependent on a unique pentasaccharide sequence located within the heparin molecule. A strategically positioned sulfate residue within the pentasaccharide sequence has a high affinity for a specific lysine site located on the AT molecule. Binding of heparin promotes a conformational change in AT which exposes an arginine situated in the AT reactive center. The exposed arginine binds covalently to specific serine centers found on certain clotting factors. This conformational change accelerates the antithrombin inhibition of procoagulant serine proteases a thousand-fold over its baseline rate. Serine proteases prone to inhibition are the clotting factors XIIa, XIa, Xa, IXa and IIa. Factor IIa, or thrombin, is the most susceptible factor.

Heparin may be used in the treatment of heart attacks and unstable angina. Heparin may be given intravenously or by injection under the skin. Other uses for its anticoagulant properties include inside blood specimen test tubes and kidney dialysis machines. In one or more embodiments, a heparin modified polymer is prepared and used to form a conductive film that can be coated on a variety of medical devices.

The heparin of one or more embodiments may have a molecular weight in a range of from 5000 Daltons to 30,000 Daltons. In one or more embodiments, the heparin has an average molecular weight of about 15,000 Daltons.

In one or more embodiments, a conductive film including a heparin modified polymer is prepared. The heparin modified polymer may include heparin covalently bound to a beta-substituted monomer having a conjugated backbone that is co-polymerized to its non-functionalized pair.

A conductive polymer comprises conjugated monomers that, once reacted to form a polymer, form a polymer that includes conjugated networks. A conjugated carbon chain has alternating single and double bonds, where the highly delocalized, polarized and electron-dense pi bonds are responsible for its electrical behavior.

Beta elimination (β-elimination) is a chemical reaction in which atoms or groups are lost from adjacent atoms, resulting in a new pi bond. One of the atoms lost is often a proton, and the new pi bond is usually formed between two carbon atoms. When the new pi bond is formed, the compound is said to be beta substituted (β-substitute). Beta-substitution is known for many conducting polymer's monomers such as poly(3,4-ethylenedioxythiophene) (PEDOT), polypyrrole (PPy), and polyaniline.

In one or more embodiments, co-polymerizing a beta-substituted monomer containing amines or carboxylic acid functional moieties with a non-functionalized monomer at 30-40 wt/wt % can form functional handles that may be used to either immobilize enzymes, such as lactase dehydrogenase, creatine deimase, lactate oxidase, and glucose oxidase, or can be used to immobilize heparin through an amide bond using dicyclohexylcarbodiimide (DCC) as an activating agent to react with the basic amine in heparin or both.

In one or more embodiments, to form the conductive film of one or more embodiments, the beta-substituted monomer comprises one or more of an amine functional moiety, a carboxylic acid functional moiety, an alcohol functional moiety, or an anhydride functional moiety. In one or more specific embodiments, the beta-substituted monomer comprises one or more of an amine functional moiety or a carboxylic acid functional moiety. In specific embodiments, the beta-substituted monomer comprises one or more of Poly(3,4-ethylenedioxythiophene) (PEDOT), polythiophene, polypyrrole, and polyaniline.

As used herein, the term “amine functional moiety” refers to a compound containing the group NR′R″, where the nitrogen atom is also bound to another group (e.g. (CH₂)ₓ, alkylene group) in the compound. In one or more embodiments R′ and R″ are independently selected from C₁₋₄ alkyl, hydrogen, or halogen.

As used herein, “alkyl,” or “alk” includes both straight and branched chain hydrocarbons, containing 1 to 20 carbons, in the normal chain, such as methyl, ethyl, propyl, isopropyl, butyl, t-butyl, isobutyl, pentyl, hexyl, isohexyl, heptyl, 4,4-dimethylpentyl, octyl, 2,2,4-trimethyl-pentyl, nonyl, decyl, undecyl, dodecyl, the various branched chain isomers thereof, and the like. Such groups may optionally include up to 1 to 4 substituents.

As used herein, “halogen” refers to one or more of a group of elements in the periodic table, more particularly fluorine (F), chlorine (Cl), bromine (Br), iodine (I), and astatine (At).

As used herein, the term “carboxylic acid functional moiety” refers to an organic moiety that contains a carboxyl (C═O) functional group and a hydroxyl (—OH) functional group.

As used herein, the term “alcohol functional moiety” refers to a moiety containing a hydroxyl (—OH) group.

As used herein, the term “anhydride functional moiety” refers to an organic moiety characterized by two acyl groups joined by an oxygen atom.

As used herein, the term “non-functionalized pair” refers to a conjugated monomer that is not beta-functionalized and is subsequently reacted with a beta-substituted monomer to form a conductive polymer. In one or more embodiments, the non-functionalized pair comprises a non-functionalized monomer selected from pyrrole, aniline, 3,4-Ethylenedioxythiophene, or thiophene.

In one or more embodiments, the non-functionalized monomer is present in an amount in the range of from 30 wt. % to 40 wt. % of the beta-substituted monomer.

In one or more embodiments, the heparin modified polymer including heparin covalently bound to a beta-substituted monomer having a conjugated backbone that is co-polymerized to its non-functionalized pair forms a conductive film that has a thickness in a range of from 50 nm to 100 μm.

In one or more embodiments, the heparin modified polymer including heparin covalently bound to a beta-substituted monomer having a conjugated backbone that is co-polymerized to its non-functionalized pair has a conductivity in a range of from 2 S/cm to 10 S/cm.

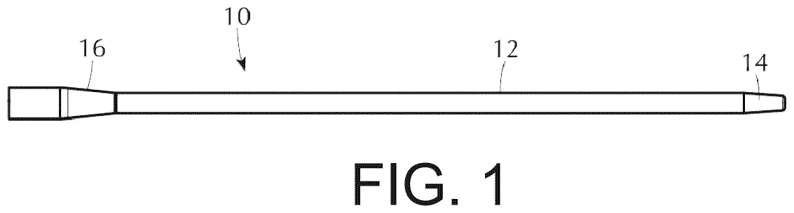

In FIG. 1, an exemplary medical device in the form of a catheter is illustrated. Tubing coated with the conductive film of one or more embodiments forms the catheter, which is shaped as needed to receive other components for forming vascular access devices. Catheter 10 comprises a primary conduit 12, which is tubing in its as-extruded form. At a distal end, a tip 14 is formed by a tipping process. At a proximal end, a flange 16 is formed as needed for receipt of other components including but not limited to catheter adapters. Exemplary vascular access devices may include a needle further to the catheter for access to blood vessels.

In one or more embodiments, the medical device is in the form of a catheter, an extension, an IV tubing, a catheter adapter, a luer port, a connector body, a device housing, a component thereof, or a combination thereof. In some embodiments, the catheter comprises a peripherally inserted central catheter (PICC), a peripheral intravenous catheter (PIVC), or a central venous catheter (CVC).

In one or more embodiments, the medical device comprises a coating including the conductive film of one or more embodiments having the heparin modified polymer. In one or more embodiments, the heparin modified polymer comprises heparin covalently bound to a beta-substituted monomer having a conjugated backbone that is co-polymerized to its non-functionalized pair. In some embodiments, because of the conductive film, the medical device passively reduces thrombus formation.

The heparin of one or more embodiments may have a molecular weight in a range of from 5000 Daltons to 30,000 Daltons. In one or more embodiments, the heparin has an average molecular weight of about 15,000 Daltons.

In one or more embodiments, as described above, to form the conductive film of one or more embodiments, the beta-substituted monomer comprises one or more of an amine functional moiety, a carboxylic acid functional moiety, an alcohol functional moiety, or an anhydride functional moiety. In one or more specific embodiments, the beta-substituted monomer comprises one or more of an amine functional moiety or a carboxylic acid functional moiety. In specific embodiments, the beta-substituted monomer comprises one or more of Poly(3,4-ethylenedioxythiophene) (PEDOT), polythiophene, polypyrrole, and polyaniline.

In one or more embodiments, the non-functionalized pair comprises a non-functionalized monomer selected from pyrrole, aniline, 3,4-Ethylenedioxythiophene, or thiophene.

In one or more embodiments, the non-functionalized monomer is present in an amount in the range of from 30 wt. % to 40 wt. % of the beta-substituted monomer.

Other embodiments are directed to methods of manufacturing a heparin modified polymer on a conductive film. In one or more embodiments, a beta-substituted monomer is cop-polymerized with a non-functionalized monomer to form a polymer on a conductive film. The polymer is then reacted with N,N′-Dicyclohexylcarbodiimide (DCC) and acetic acid to form an anhydride of the polymer on the conductive film. The anhydride is reacted with heparin to form the heparin modified polymer on the conductive film.

In one or more embodiments, the co-polymerization of the beta-substituted monomer and the non-functionalized monomer is performed in the presence of at least one counter electrode and a voltage source. In some embodiments, the at least one counter electrode comprises one or more of indium tin oxide (ITO), platinum (Pt), silver (Ag), iron, aluminum, copper, palladium, mercurous chloride, and silver chloride (AgCl).

In other embodiments, the co-polymerization is a chemical co-polymerization using a strong oxidizer.

The heparin of one or more embodiments may have a molecular weight in a range of from 5000 Daltons to 30,000 Daltons. In one or more embodiments, the heparin has an average molecular weight of about 15,000 Daltons.

In one or more embodiments, the beta-substituted monomer comprises one or more of an amine functional moiety, a carboxylic acid functional moiety, an alcohol functional moiety, or an anhydride functional moiety. In one or more specific embodiments, the beta-substituted monomer comprises one or more of an amine functional moiety or a carboxylic acid functional moiety. In specific embodiments, the beta-substituted monomer comprises one or more of Poly(3,4-ethylenedioxythiophene) (PEDOT), polythiophene, polypyrrole, and polyaniline.

In one or more embodiments, the non-functionalized pair comprises a non-functionalized monomer selected from pyrrole, aniline, 3,4-Ethylenedioxythiophene, or thiophene.

In one or more embodiments, the non-functionalized monomer is present in an amount in the range of from 30 wt. % to 40 wt. % of the beta-substituted monomer.

In one or more embodiments, the heparin modified polymer including heparin covalently bound to a beta-substituted monomer having a conjugated backbone that is co-polymerized to its non-functionalized pair forms a conductive film that has a thickness in a range of from 50 nm to 100 μm.

In one or more embodiments, the heparin modified polymer including heparin covalently bound to a beta-substituted monomer having a conjugated backbone that is co-polymerized to its non-functionalized pair has a conductivity in a range of from 2 S/cm to 10 S/cm.

In one or more embodiments, the heparin modified polymer is then removed from the conductive film. The heparin modified polymer may be removed from the conductive film by any suitable method known to the skilled artisan. In one or more embodiments, removing the heparin modified polymer from the conductive film comprises exposing the heparin modified polymer and the conductive film to double sided tape.

In one or more embodiments, without intending to be bound by theory, it is thought that the heparinized conductive film could inhibit thrombin (Factor IIa) and inhibit fibrin stabilizing factors in the vasculature. The conductive substrate could then be used to interrogate different analytes or vitals in the blood stream such as blood pressure, blood temperature, or through dopant exchange Cl or HCO3-. In one or more embodiments, a simple pattern design on the outside of a catheter could be done through first chemical vapor deposition and standard photolithographic techniques of a conductive substrate followed by chemical and electrochemical paradigm or through a roll-transfer method to transfer the synthesized pre-cursor film (i.e., without the heparin functionalization) onto a catheter and then subsequently react with heparin as described above.

The disclosure is now described with reference to the following examples. Before describing several exemplary embodiments of the disclosure, it is to be understood that the disclosure is not limited to the details of construction or process steps set forth in the following description. The disclosure is capable of other embodiments and of being practiced or being carried out in various ways.

### EXAMPLES

### Example 1

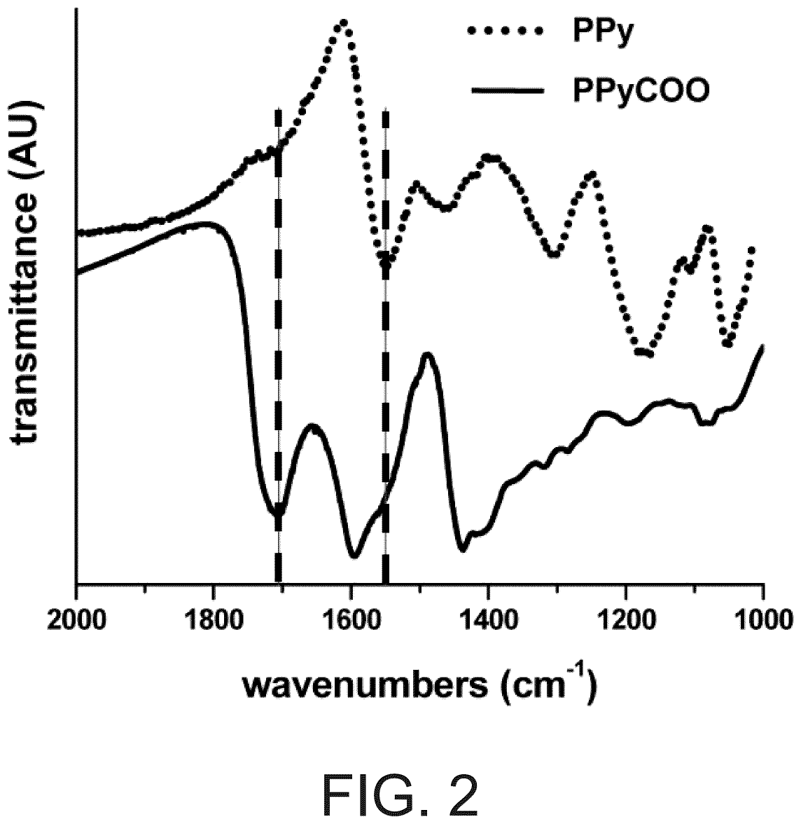

Pyrrole 3-carboxylic acid was electrochemically synthesized with unmodified purified pyrrole at three levels (30, 35, 40 wt/wt %). This was done by placing an anode comprised on indium tin oxide electrode, with a counter-electrode made of platinum, and a reference electrode comprised of a Ag/AgCl electrode. The aqueous electrochemical reaction was performed by sweeping from −1 to 1.5 V at a scan rate of 10 mV/s for a total charge of 250 mC resulting in a 1 μm thick film in the presence of 0.1 M NaCl. This film was then washed. Confirmation of carboxylic acid functionalization was done using FTIR (FIG. 2). The strong band at 1703 cm-1 of PPyCOOH (solid line) confirmed the presence of carboxylic acid (—COOH) functionality in bulk. The two vertical dashed lines at 1703 cm⁻¹ and 1548 cm⁻¹ represent the positions of carboxylic acid and second amine (from the Ppy backbone) respectively.

After the film was synthesized, it was placed in a 100 ml beaker containing water. 0.1 M DCC and 0.1 M acetic acid were added to the beaker with stirring to make an anhydride after 4 hours of reaction at room temperature. The film was then removed, rinsed with DI water, and then placed in another beaker containing water and Heparin (100 mg/mL) at 1 M concentration. The reaction was allowed to occur overnight. After the reaction was complete, several rinses with DDI water was performed. Confirmation of attachment of heparin onto the surface was done by SEM (EDX) to identify an increase in % S on the surface of the film (Table 1).

TABLE 1

| Sample | % C | % O | % N | % S |
|---|---|---|---|---|
| Polypyrrole- | 62.4 | 25 | 12.6 | 0 |
| COOH | | | | |
| Polypyrrole- | 50.8 | 40 | 6.3 | 2.71 |
| Heparin | | | | |

Removal of PPy films from conductive ITO was performing using double sided tape to remove the modified PPy-Heparin film from the conductive substrate and tape onto an insulating slide. Using silver epoxy leads were placed and resistance measured of the conductive substrate of PPy-PPy(COOH) with and without Heparin modifications (Table 2).

TABLE 2

| Condition | Resistance (kΩ) | Conductance |
|---|---|---|
| Ppy-COOH | 1.18E+00 | 8.5 |
| Ppy-Heparin | 1.27E+00 | 7.9 |
| Tape | 1.85E+07 | 0.00000054 |

Reference throughout this specification to “one embodiment,” “certain embodiments,” “one or more embodiments” or “an embodiment” means that a particular feature, structure, material, or characteristic described in connection with the embodiment is included in at least one embodiment of the invention. Thus, the appearances of the phrases such as “in one or more embodiments,” “in certain embodiments,” “in one embodiment” or “in an embodiment” in various places throughout this specification are not necessarily referring to the same embodiment of the invention. Furthermore, the particular features, structures, materials, or characteristics may be combined in any suitable manner in one or more embodiments.

Although the invention herein has been described with reference to particular embodiments, it is to be understood that these embodiments are merely illustrative of the principles and applications of the present invention. It will be apparent to those skilled in the art that various modifications and variations can be made to the method and apparatus of the present invention without departing from the spirit and scope of the invention. Thus, it is intended that the present invention include modifications and variations that are within the scope of the appended claims and their equivalents.

1. A conductive film comprising a heparin modified polymer, the heparin modified polymer comprising heparin covalently bound to a beta-substituted monomer having a conjugated backbone that is co-polymerized to its non-functionalized pair.

2. The conductive film of claim 1, wherein the beta-substituted monomer comprises one or more of an amine functional moiety or a carboxylic acid functional moiety.

3. The conductive film of claim 1, wherein the beta-substituted monomer comprises one or more of an amine functional moiety, carboxylic acid functional moiety, an alcohol functional moiety or an anhydride functional moiety.

4. The conductive film of claim 1, wherein the beta-substituted monomer comprises one or more of PEDOT, polythiophene, polypyrrole, and polyaniline.

5. The conductive film of claim 1, wherein the non-functionalized pair comprises a non-functionalized monomer selected from pyrrole, aniline, 3,4-Ethylenedioxythiophene, thiophene.

6. The conductive film of claim 5, wherein the non-functionalized monomer is present in an amount in a range of 30 wt. % to 40 wt. % of the beta-substituted monomer.

7. The conductive film of claim 1, wherein the heparin has a molecular weight in a range of 5000 Daltons to 30,000 Daltons.

8. The conductive film of claim 1, wherein the conductive film has a thickness in a range of from 50 nm to 100 μm.

9. The conductive film of claim 1, wherein the heparin modified polymer has a conductivity in a range of from 2 S/cm to 10 S/cm.

10. A medical device having a coating comprising the conductive film of claim 1.

11. A method of manufacturing a heparin modified polymer on a conductive film, the method comprising:
co-polymerizing a beta-substituted monomer with a non-functionalized monomer to form a polymer on a conductive film;
reacting the polymer with N,N′-Dicyclohexylcarbodiimide (DCC) and acetic acid to form an anhydride of the polymer on the conductive film; and
reacting the anhydride with heparin to form the heparin modified polymer on the conductive film.

12. The method of claim 11, wherein the co-polymerization is performed in the presence of at least one counter electrode and a voltage source, and wherein the at least one counter electrode comprises one or more of indium tin oxide (ITO), platinum (Pt), silver (Ag), iron, aluminum, copper, palladium, mercurous chloride, and silver chloride (AgCl).

13. The method of claim 11, wherein the co-polymerization is chemical co-polymerization using a strong oxidizer.

14. The method of claim 11, wherein the beta-substituted monomer comprises one or more of an amine functional moiety, carboxylic acid functional moiety, an alcohol functional moiety or an anhydride functional moiety, or wherein the beta-substituted monomer comprises one or more of PEDOT, polypyrrole, polythiophene, and polyaniline.

15. The method of claim 11, wherein the non-functionalized monomer comprises one or more of pyrrole, aniline, 3,4-Ethylenedioxythiophene, thiophene, and wherein the non-functionalized monomer is present in an amount in a range of 30 wt. % to 40 wt. % of the beta-substituted monomer.

16. The method of claim 11, wherein the heparin has a molecular weight in a range of 5000 Daltons to 30,000 Daltons, and wherein the heparin modified polymer has a conductivity in a range of from 2 S/cm to 10 S/cm.

17. The method of claim 11, wherein the conductive film has a thickness in a range of from 50 nm to 100 μm.

18. The method of claim 11, further comprising removing the heparin modified polymer from the conductive film.

19. The method of claim 18, wherein removing the heparin modified polymer from the conductive film comprises exposing the heparin modified polymer and the conductive film to double sided tape.

20. The method of claim 11, further comprising coating the heparin modified polymer on a body of a medical device, wherein the medical device is in the form of a catheter, an extension, an IV tubing, a catheter adapter, a luer port, a connector body, a device housing, a component thereof, or a combination thereof.
